(19) United States
(12) Patent Application Publication
(10) Pub. No.: US 2025/0318741 A1
(43) Pub. Date: Oct. 16, 2025

(54) LIGHT TRANSMISSION SUBSTRATES AND LIGHT TRANSMISSION SUBSTRATE POSITIONING STRUCTURES

(71) Applicant: Plethys LLC, San Diego, CA (US)

(72) Inventor: Andrew Gladue, San Diego, CA (US)

(21) Appl. No.: 18/922,958

(22) Filed: Oct. 22, 2024

Related U.S. Application Data
(60) Provisional application No. 63/633,542, filed on Apr. 12, 2024.

(51) Int. Cl. A61B 5/024; A61B 5/00

(52) U.S. Cl. CPC A61B 5/02427; A61B 5/6801; A61B 2560/0462; A61B 2562/0233

(57) ABSTRACT

Methods, devices, and systems are described for light transmission substrates and light transmission substrate positioning structures. The light transmission substrate includes a transparent resin having a first side and a second side opposite the first side. The first side is configured to cover a plurality of light emitters and a light sensor at a wearable biometric tracker. The first side is configured to receive light from the plurality of light emitters at the wearable biometric tracker. The second side is configured to interface with a user skin tissue. The second side configured to pass light received by the transparent resin to the user skin tissue. The light transmission substrate is configured to pass light from the plurality of light emitters to the user skin tissue in response to the light transmission substrate being positioned between the wearable biometric tracker and the user skin tissue.

### CROSS-REFERENCE TO RELATED APPLICATIONS

This application claims priority to and the benefit of the earlier filing date of U.S. Provisional Patent Application Ser. No. 63/633,542, filed Apr. 12, 2024, the contents of which are fully incorporated by reference herein in its entirety.

### TECHNICAL FIELD

The subject matter described herein generally relates to light transmission substrates, and more particularly, to light transmission substrates and light transmission substrate positioning structures.

### BACKGROUND

The use of portable and wearable biometric trackers such as smartwatches, fitness trackers, smart jewelry, and so forth, is ubiquitous. These devices vary in complexity and capability and are customized for different uses across various industries. Sensors embedded in these devices track body movements and record various physiological parameters (e.g., heart rate, blood pressure, body temperature, sleep patterns) of users. Various user characteristics, however, may impede the light received at these sensors. For example, tattoos, excessive body hair, or dark skin obstructions may partially prevent or entirely prevent light received by the wearable biometric tracker sensor. Additionally, tattoos, excessive body hair, or dark skin obstructions may prohibit light emitted from one or more light sources from contacting or penetrating the user's skin. The obstructed light transmission between the light sources and the corresponding sensors results in nonexistent or inaccurate measurements by the sensors. In turn, portable and wearable biometric trackers cannot generate accurate measurements based on nonexistent or inaccurate measurements.

As such, wearable biometric trackers and supporting structures that resolve these deficiencies and enable the accurate determination of physiological parameters are needed.

### SUMMARY

The present disclosure relates generally to the fields of light transmission substrates and light transmission substrate positioning structures.

In one aspect, disclosed herein is a transparent resin comprising a first side and a second side opposite the first side. The first side is configured to cover a plurality of light emitters and a light sensor at a wearable biometric tracker. The first side is configured to receive light from the plurality of light emitters at the wearable biometric tracker. The second side is configured to interface with a user skin tissue. The second side configured to pass light received by the transparent resin to the user skin tissue. The transparent resin is configured to pass light from the plurality of light emitters to the user skin tissue with the transparent resin positioned between the wearable biometric tracker and the user skin tissue.

In some variations, the transparent resin has a viscosity value in a range of 500 to 10,000 centipoise (cps). Furthermore, the transparent resin has a greater thickness at an inner portion covering the plurality of light emitters and the light sensor than an outer portion of the transparent resin. Additionally, the inner portion is made of a first material and wherein the outer portion are made of a second material. Furthermore, the second side is configured to receive at least one of reflected light or transmitted light from the user skin tissue and the first side is configured to pass reflected light received by the user skin tissue to the wearable biometric tracker.

In some variations, the transparent resin is configured to pass the reflected light from the user skin tissue to the light sensor of the wearable biometric tracker with the transparent resin situated between the wearable biometric tracker and the user skin tissue. Additionally, the second side of the transparent resin has at least one of a convex geometry or a dome-shaped protrusion. Furthermore, the transparent resin has an index of refraction in a range of approximately 1.4 to 1.7.

In some variations, the transparent resin has an index of refraction in a range of approximately 1.5 to 1.56. Furthermore, the light sensor of the wearable biometric tracker is a photoplethysmography (PPG) sensor and an LED is at least one light emitter of the plurality of light emitters. Additionally, the transparent resin is configured to perform at least one of refract or redirect light passing through the transparent resin.

In another aspect, disclosed herein is a device having a light transmission substrate including two or more transparent layers with a first transparent layer disposed over a second transparent layer. The first transparent layer is configured to cover a plurality of light emitters and a light sensor at a wearable biometric tracker. The first transparent layer is configured to receive light from the plurality of light emitters at the wearable biometric tracker. The second transparent layer is configured to interface with a user skin tissue. The second transparent layer is configured to pass light received by the light transmission substrate to the user skin tissue. The device includes a casing configured to suspend the two or more transparent layers between the wearable biometric tracker and the user skin tissue. The light transmission substrate is configured to pass light from the plurality of light emitters to the user skin tissue with the light transmission substrate positioned between the wearable biometric tracker and the user skin tissue.

In some variations, the casing further comprises two or more elastic loops configured to suspend the two or more transparent layers between the wearable biometric tracker and the user skin tissue. Furthermore, the two or more elastic loops are situated at opposing ends of the casing. Additionally, the two or more elastic loops are parallel to each other. Furthermore, the two or more elastic loops are parallel to each other and to the opposing ends of the casing. Additionally, the casing covers an outer edge of the light transmission substrate while leaving an exposed center portion of the light transmission substrate.

In some variations, one transparent layer of the two or more transparent layers has a height dimension that extends beyond the casing. Additionally, the other transparent layer of the two or more transparent layers has the outer edge covered by the casing. Furthermore, the light transmission substrate includes three or more transparent layers. Additionally, the second transparent layer is configured to receive at least one of reflected light or transmitted light from the user skin tissue and the first transparent layer is configured to pass reflected light received by the user skin tissue to the wearable biometric tracker. Furthermore, the light transmission substrate is configured to pass the at least one of the reflected light or the transmitted light from the user skin tissue to the light sensor of the wearable biometric tracker with the light transmission substrate situated between the wearable biometric tracker and the user skin tissue.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1A illustrates a view of a back portion of a wearable biometric tracker such as a smartwatch including a plurality of light emitters and a light sensor;

FIG. 1B illustrates a light transmission substrate that is configured to adhere to the back of a wearable biometric tracker and cover the plurality of light emitters and the light sensor;

FIG. 1C depicts a light transmission substrate attached to the back of a wearable biometric tracker that covers the plurality of light emitters and the light sensor of the wearable biometric tracker;

FIG. 2 depicts a block diagram illustrating an emitted light transmission between a wearable biometric tracker (e.g., a watch), the domed light transmission substrate and the skin of a user;

FIG. 3A depicts an example of a wearable biometric tracker enclosure in a closed state;

FIG. 3B depicts an example of a wearable biometric tracker enclosure in an open state;

FIG. 3C depicts an example of a front plate configured to protect the front side of the wearable biometric tracker;

FIG. 3D depicts an example of a back plate configured to protect the back side of the wearable biometric tracker;

FIG. 4A depicts a front view of an example of a wearable biometric tracker enclosure configured to suspend the light transmission substrate against the wearable biometric tracker;

FIG. 4B depicts a back view of an example of a wearable biometric tracker enclosure configured to suspend the light transmission substrate against the wearable biometric tracker;

FIG. 5 depicts another view of the wearable biometric tracker enclosure configured to suspend the light transmission substrate against the wearable biometric tracker;

FIG. 6A depicts a representation of a back of the wearable biometric tracker;

FIG. 6B depicts an example of a light transmission substrate having a plurality of protrusions that are configured to pass light through the resin substrate;

FIG. 6C shows an example of the transparent resin substrate superimposed over the backside of the wearable biometric tracker;

FIG. 7 depicts an example of a detachable light transmission substrate configured to cover the light sensor and the plurality of light emitters using a pair of loops;

FIG. 8 depicts multiple graphical representations that include data obtained by various sensors of a Garmin smartwatch without applying the light transmission substrate;

FIG. 9 depicts multiple graphical representations that include data obtained by various sensors of a Garmin smartwatch with the application of the light transmission substrate;

FIG. 10A depicts a graphical representation that includes a comparison of the operation of an Apple Watch without the light transmission substrate to the operation with the light transmission substrate;

FIG. 10B depicts a graphical representation that shows the manner in which an Apple fitness tracker operates when applying the wearable biometric tracker enclosure and the wearable biometric tracker enclosure;

FIG. 11 depicts a top view of an optical lens casing that partially encloses a light transmission substrate;

FIG. 12 depicts a side view of an optical lens casing that partially encloses a light transmission substrate;

FIG. 13 depicts the light transmission substrate configured to be coupled to the partially enclosed by the optical lens casing having elastic loops;

FIGS. 14-16 depict a method of applying the optical lens casing to a fitness tracker having straps;

FIG. 17 depicts another light transmission substrate configured to be coupled to the partially enclosed by the optical lens casing having elastic loops;

FIG. 18 depicts another light transmission substrate configured to be coupled to the partially enclosed by the optical lens casing having elastic loops;

FIG. 19 depicts another light transmission substrate configured to be coupled to the partially enclosed by the optical lens casing having elastic loops;

FIG. 20 depicts a perspective view of a tray having a plurality of mold wells configured to form a plurality of light transmission substrates;

FIG. 21 depicts a perspective view of a tray having a plurality of mold wells configured to form a plurality of light transmission substrates;

FIG. 22A depicts a cross-sectional view of the tray in which the cross-section is made along the length of the tray;

FIG. 22B depicts a cross-sectional view of the tray in which the cross-section is made along the width of the tray;

FIG. 23 depicts a perspective view of a three-layered light transmission substrate;

FIG. 24 depicts a top view of a three-layered light transmission substrate;

FIG. 25A depicts a side view of a three-layered light transmission substrate along a length of the light transmission substrate; and

FIG. 25B depicts a side view of the three-layered light transmission substrate along a length of the light transmission substrate.

Like reference numbers and designations in the various drawings indicate like elements.

### DETAILED DESCRIPTION

The methods, systems, and apparatuses described herein are for light transmission substrate and substrate positioning structures. In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

The use of portable and wearable biometric trackers such as smartwatches, fitness trackers, smart jewelry, and so forth, is ubiquitous. These devices vary in complexity and capability and are customized for different uses across the healthcare, fitness, and entertainment industries. Wearable biometric trackers utilize embedded sensors, namely photoplethysmography (PPG) sensors, to track body movements and determine various physiological parameters of consumers, e.g., heart rate, blood pressure, body temperature, sleep patterns, and so forth. These sensors can include a light source that emits light on an exterior surface of tissue (e.g., a consumer's wrist) and a photodetector that measures the light reflected from the tissue. The measured light is subsequently utilized to determine a number of physiological parameters.

As stated above, various consumer characteristics, however, may obstruct the emitted light from contacting or penetrating the surface of tissue, e.g., a wrist of a user. For example, obesity, tattoos, excessive body hair, or dark skin obstructions can prevent or obstruct the emitted light from correctly reaching the surface of a user's wrist, resulting in inaccurate operation of the PPG sensors. Consequently, physiological data may not be accurately determined.

The light transmission substrate as described in the present disclosure, when adhered to various wearable biometric trackers, addresses and resolves light transmission deficiencies previously described. Specifically, the light transmission substrate as described herein, when detachably adhered to the back portions of the wearable biometric trackers (e.g., a smartwatch, a fitness tracker), enables light emitted from the PPG sensors installed in these devices to be accurately directed towards the skin on, e.g., a user's wrist. In this way, the light transmission substrate serves as a light guide or sensor operation guide that improves the accuracy with which wearable biometric trackers obtain data for determining physiological parameter values of users.

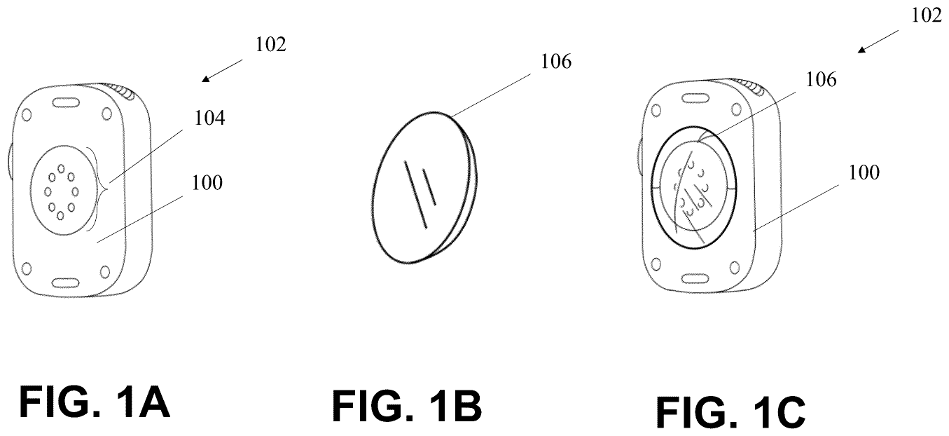

FIG. 1A illustrates a view of a back portion 100 of a wearable biometric tracker 102 (such as a smartwatch) including a light emitter and an array of sensors. The back portion 100 of the wearable biometric tracker 102 may include a plurality of light emitters 104 and a light sensor. The light sensor may be configured to measure incoming light to determine various physiological parameters (e.g., heart rate, blood pressure) of a consumer or user.

The back portion 100 also includes a plurality of light emitters 104 configured to emit light at different wavelengths and colors. Each light emitter of the plurality of light emitters 104 is configured to operate at a wavelength and/or frequency optimized for detecting user features and/or penetrating different layers of tissue. For example, green lasers at the plurality of emitters 104 are suitable for detecting motion but inadequate for total skin penetration. In another example, red lasers are inadequate for detecting motion but suitable for total skin penetration. In some embodiments, the light sensor may be a photodiode that is situated proximate to the plurality of light emitters 104. In some embodiments, the plurality of light emitters 104 are LEDs.

In some embodiments related to wearable biometric trackers 102, the plurality of light emitters 104 may emit light that is configured to contact and penetrate the user skin. Different frequencies and colors of light may have different absorption and penetration rates and depths as the light travels through different tissues and blood. Additionally, different frequencies and colors of light may have different reflection and transmission rates as light travels back through the different tissues to the light sensor at the wearable biometric tracker 102. This light transmission pathway allows for different physiological features of the user to be determined by the wearable biometric tracker 102 using the light sensor. For example, the veins carrying blood in the skin can absorb different amounts of light depending on the amount of blood in the vein and the pulsation cycle. More light may be absorbed when there is more blood in the vein and less light may be absorbed when there is less blood in the vein. The light sensor may be configured to detect the varying amounts of light reflected back from the pulsing vein to determine the user heartbeat.

FIG. 1B illustrates a domed light transmission substrate 106 that is configured to adhere to the back of a wearable biometric tracker 102 and cover the plurality of light emitters 104 and the light sensor. The domed light transmission substrate 106 may be a light transmission medium configured to refract or redirect light emitted from the plurality of light sources 104 of the wearable biometric tracker 102 onto a surface of tissue, e.g., a wrist of a user wearing the wearable biometric tracker 102. Similarly, the domed light transmission substrate 106 may be configured to be a light guide to allow reflected or transmitted light in the skin tissues to pass from the skin to the light sensor.

The domed light transmission substrate 106 may be configured to cover the light sensor and the plurality of light emitters 104. In some embodiments, the domed light transmission substrate 106 may be thicker at the portions covering the light sensor and the light emitters 104 relative to other portions of the domed light transmission substrate 106 covering other portions of the back portion 100. In some embodiments, the domed light transmission substrate 106 may be convex or have a dome shape at the portion covering the light sensor and the plurality of light emitters 104. In some embodiments, the domed light transmission substrate 106 may be made of two materials in which a first material covers the light sensor and a second material covers the plurality of light emitters 104.

In some embodiments, the domed light transmission substrate 106 can be translucent and formed of one or more of silicone, polycarbonate, polymethyl methacrylate (PMMA), epoxy resin, glass, and other comparable substances. The domed light transmission substrate 106 may have a circular or spherical shape and can include a convex or concave geometry. In some embodiments, the domed light transmission substrate 106 can be formed in a number of other shapes (e.g., rectangle, square), including a shape that conforms to the back portion 100 of a particular wearable biometric tracker 102. Further, the domed light transmission substrate 106 may have a viscosity value in a range of 500 to 10,000 centipoise (cps).

FIG. 1C depicts a domed light transmission substrate 106 attached to the back of a wearable biometric tracker 102 that covers the plurality of light emitters 104 and the light sensor of the wearable biometric tracker 102. The domed light transmission substrate 106 has a circular or spherical shape with at least one portion with a convex or concave geometry.

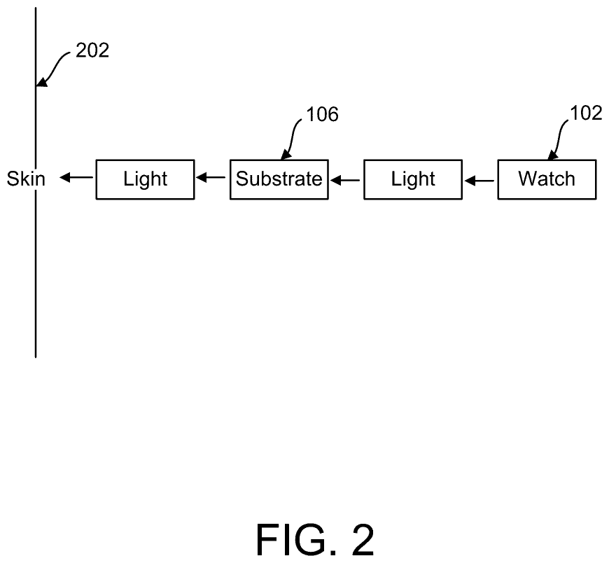

FIG. 2 depicts a block diagram illustrating an emitted light transmission between a wearable biometric tracker 102 (e.g., a watch), the domed light transmission substrate 106 and the skin of a user. The light may be emitted from the plurality of light emitters 104 at the wearable biometric tracker 102. The wearable biometric tracker 102 may be configured to emit light that enters the light transmission substrate 106. The domed light transmission substrate 106 may be configured to refract or redirect light entering the light transmission substrate 106. The refracted or the redirected light may be configured to exit the domed light transmission substrate 106 to the user skin 202. The refracted or redirected light exiting the domed light transmission substrate 106 may be configured to contact or penetrate the user skin 202.

The domed light transmission substrate 106 may be a bi-directional light transmission guide. That is, the domed light transmission substrate 106 may be configured to refract or redirect light entering the domed light transmission substrate 106 from either side. In some embodiments, reflected or transmitted light from the user skin may enter the light transmission substrate 106. The domed light transmission substrate 106 may be configured to refract or redirect light entering the light transmission substrate 106. The refracted or the redirected light may be configured to exit the domed light transmission substrate 106 towards the light sensor at the wearable biometric tracker 102. The exiting refracted or redirected light may be configured to be received at the light sensor of the wearable biometric tracker 102.

In some embodiments, the user skin 202 can include tattoos, dark skin obstructions, excessive body hair, and/or be associated with an obese individual. In some embodiments, the domed light transmission substrate 106 has an index of refraction domed light transmission substrate 106 in a range of approximately 1.4 to 1.7 or in a range of approximately 1.5 to 1.56. These index of refraction ranges enable the domed light transmission substrate 106 to refract or redirect the light emitted from the wearable biometric tracker 102. The domed light transmission substrate 106 may be configured to maximize the light contact and penetration of the surface of the skin 202, including skin that may include tattoos, excessive body hair, dark skin obstructions, and/or be associated with an obese individual. This increased light transmission between the skin and the wearable biometric trackers 102 may resolve the light-obscuring impediments related to excessive body hair, dark skin obstructions, or being obese.

In some embodiments, the domed light transmission substrate 106 may be adhered to the back portion 100 of the wearable biometric tracker 102. In some embodiments, the wearable biometric tracker 102 may apply straps that selectively couple together to sustain the wearable biometric tracker 102 to the user's wrist or other skin portion. The domed light transmission substrate 106 can be detachably positioned on the back portion 100 of the wearable biometric tracker 102 such that the domed light transmission substrate 106 interfaces directly with a skin 202 of the user. In some embodiments, the wearable biometric tracker 102 can include a photoplethysmography (PPG) sensor. The light emitter (e.g., a Light Emitting Diode (LED)) can emit light 204 that contacts the light transmission substrate 106, which in turn refracts the light 204 onto the skin 202 of the user. The light can then be reflected upwards from the surface of the skin 202 and detected by the photodetector of the PPG sensor. The reflected or refracted light may be an input to the PPG sensor to determine various physiological parameters associated with a user. For example, the reflected or refracted light may determine or measure blood volume changes.

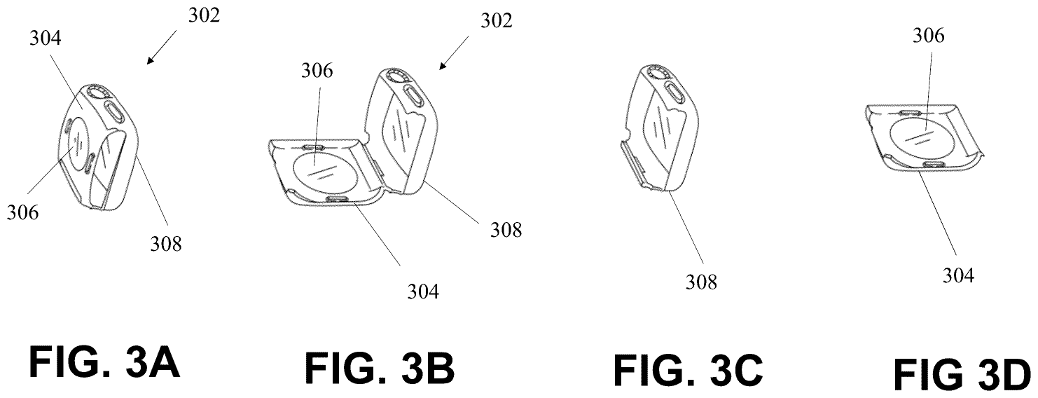

FIG. 3A depicts an example of a wearable biometric tracker enclosure 302 in a closed state. The wearable biometric tracker enclosure 302 may be configured to sustain the domed light transmission substrate 106 against the backside of the wearable biometric tracker 102. In some embodiments, the wearable biometric tracker enclosure 302 may have an integrated light transmission substrate 306 configured to interface with the backside of the wearable biometric tracker 102. The wearable biometric tracker enclosure 302 includes a front plate 308 configured to protect the front side of the wearable biometric tracker 102. The wearable biometric tracker enclosure 302 includes a back plate 304 configured to protect the back side of the wearable biometric tracker 102. The wearable biometric tracker enclosure 302 includes a hinge configured to allow the front plate 308 to rotate relative to the back plate 304. The wearable biometric tracker enclosure 302 may also include an overhanging tab to allow the front plate 308 to selectively couple to a recess or a different overhang at the back plate 304. In the closed state, the front plate 308 and the back plate 304 of the wearable biometric tracker enclosure 302 may be configured to sandwich the domed light transmission substrate 106 and the wearable biometric tracker 102 together.

FIG. 3B depicts an example of a wearable biometric tracker enclosure 302 in an open state. The back plate 304 of the wearable biometric tracker enclosure 302 may include an integrated light transmission substrate 306. In some embodiments, the back plate 304 of the wearable biometric tracker enclosure 302 may include a cavity configured to receive the light transmission substrate 106. The back plate 304 may include an obscure portion and a transparent portion 306. The integrated light transmission substrate 306 may be a light transmission medium configured to refract or redirect light emitted from the plurality of light sources 104 of the wearable biometric tracker 102 onto a surface of tissue, e.g., a wrist of a user wearing the wearable biometric tracker 102. Additionally, and/or alternatively, integrated light transmission substrate 306 may be aligned with the domed light transmission substrate 106. In some embodiments, the integrated light transmission substrate 306 of the back plate 304 may be configured to be aligned with the portion of the domed light transmission substrate 106 configured to cover the plurality of light emitters 104 and the light sensor of the wearable biometric tracker 102. In some embodiments, the integrated light transmission substrate 306 may be replaced with an aperture through which the domed light transmission substrate 106 may be inserted. In an open state, the front plate 308 and the back plate 304 are separated to allow the removal of the wearable biometric tracker 102.

FIG. 3C depicts an example of a front plate 308 configured to protect the front side of the wearable biometric tracker 102. The front portion 308 may interface with the display screen of the wearable biometric tracker 102. In some embodiments, the front plate 308 may have a shape and dimensions that enable the wearable biometric tracker 102 to be selectively inserted into the wearable biometric tracker enclosure 302. In some embodiments, the wearable biometric tracker enclosure 302 can be formed of a hard plastic material, though other comparable materials are also contemplated.

FIG. 3D depicts an example of a back plate 304 configured to protect the back side of the wearable biometric tracker 102. In some embodiments, the wearable biometric tracker enclosure 302 can include a back plate 304 with the integrated light transmission substrate 306. The integrated light transmission substrate 306 of the back plate 304 may be configured to be aligned with the domed light transmission substrate 106. In some embodiments, the integrated light transmission substrate 306 of the back plate 304 may be replaced with an aperture configured to be aligned with the portion of the domed light transmission substrate 106 configured to cover the plurality of light emitters 104 and the light sensor of the wearable biometric tracker 102. In this way, the integrated light transmission substrate 306 and/or the domed light transmission substrate 106 can be securely positioned on the back of the wearable biometric tracker 102 to facilitate the accurate redirection or refraction of a PPG signal (e.g., a light emitted by an LED of the PPG sensor of the wearable biometric tracker 102) onto an outer surface of the skin 202 of a user, irrespective of one or more obstructions present on the skin, e.g., tattoos, excessive body hair, and so forth.

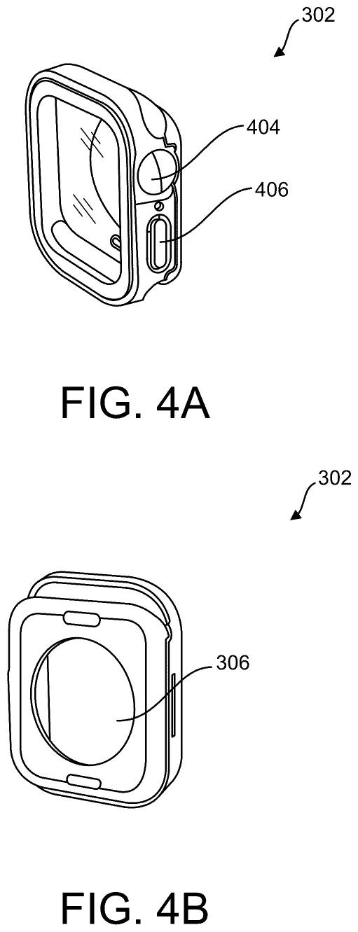

FIG. 4A depicts a front view of an example of a wearable biometric tracker enclosure 302 configured to suspend the integrated light transmission substrate 306 against the wearable biometric tracker 102. The wearable biometric tracker enclosure 302 includes a front plate 308 including an aperture 404 and a pushbutton 406. The aperture 404 includes a cutout that allows the user to select a button or rotate a knob or dial at the wearable biometric tracker 102. In some aspects, the pushbutton 406, when depressed by a user, can engage a button on the wearable biometric tracker 102. In some embodiments, the wearable biometric tracker enclosure 302 may include a button for selectively decoupling the front side from the back side of the wearable biometric tracker enclosure 302 to place the wearable biometric tracker enclosure 302 in an open state. In some aspects, when the wearable biometric tracker enclosure 302 is positioned on and secured to the wearable biometric tracker 102, any knobs, pushbuttons, and/or other interactive components can protrude from the aperture 404 such that the user can easily interact with these components.

FIG. 4B depicts a back view of an example of a wearable biometric tracker enclosure 302 configured to suspend the integrated light transmission substrate 306 against the wearable biometric tracker 102. The back plate 304 of the wearable biometric tracker enclosure 302 includes the integrated light transmission substrate 306 with which the plurality of light emitters 104 may be aligned. In some embodiments, the integrated light transmission substrate 306 may be an aperture into which the domed light transmission substrate 106 may be deposited. This means that user skin may press against the domed light transmission substrate 106 while the wearable biometric tracker enclosure 302 suspends the domed light transmission substrate 106. The transparent portion 306 of the back plate 304 may be configured to be aligned with the domed light transmission substrate 106. In some embodiments, the integrated light transmission substrate 306 of the back plate 304 may be configured to cover the plurality of light emitters 104 and the light sensor of the wearable biometric tracker 102. In some embodiments, integrated light transmission substrate 306 of the back plate 304 may be an aperture.

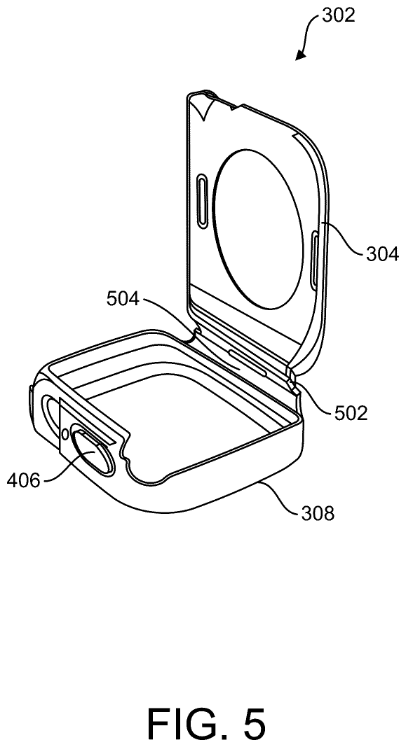

FIG. 5 depicts another view of the wearable biometric tracker enclosure 302 configured to suspend the domed light transmission substrate 106 against the wearable biometric tracker 102. The wearable biometric tracker enclosure 302 includes a front plate 308 configured to protect the front side of the wearable biometric tracker 102. The wearable biometric tracker enclosure 302 includes a back plate 304 configured to protect the back side of the wearable biometric tracker 102. The wearable biometric tracker enclosure 302 includes a hinge configured to allow the front plate 308 to rotate relative to the back plate 304. The wearable biometric tracker enclosure 302 may also include an overhanging tab to allow the front plate 308 to selectively couple to a recess or a different overhang at the back plate 304. In the closed state, the front plate 308 and the back plate 304 of the wearable biometric tracker enclosure 302 may be configured to sandwich the domed light transmission substrate 106 and the wearable biometric tracker 102 together. In the open position 502, the domed light transmission substrate 106 can be detached and removed from the wearable biometric tracker 102 and the wearable biometric tracker 102 can be charged.

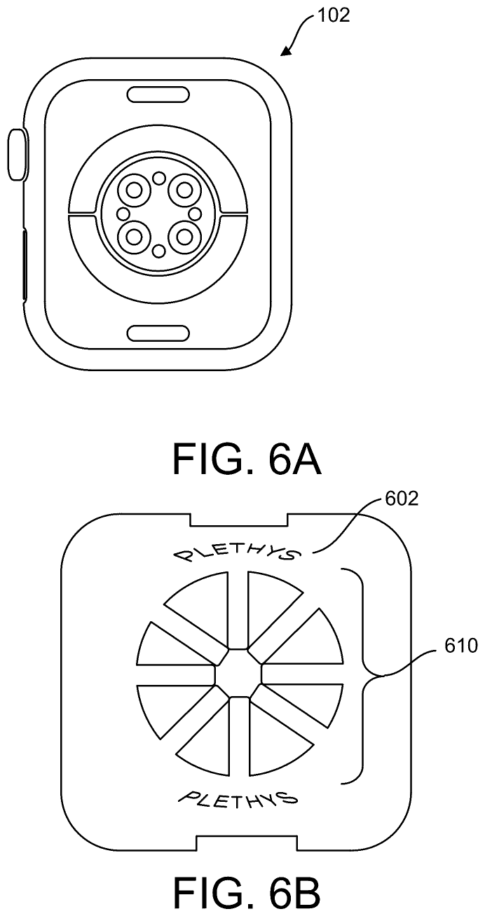

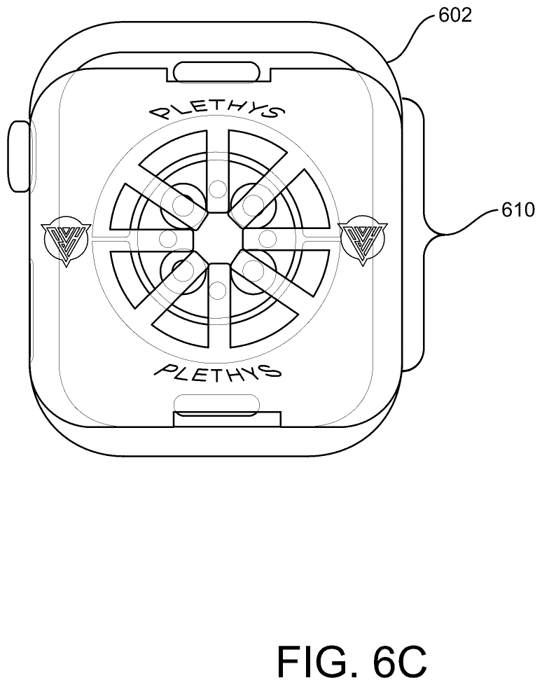

FIG. 6A depicts a representation of a back of the wearable biometric tracker 102. The back of the wearable biometric tracker 102 may include the plurality of light emitters 104 and the light sensor. Additionally, the back of the wearable biometric tracker 102 may include a band release button, an electrical heart sensor, speaker vents, air vents, a blood oxygen sensor, and an optical heart sensor.

FIG. 6B depicts an example of a spoked light transmission substrate 602 having a plurality of protrusions 610 that are configured to pass light through the resin substrate. In some embodiments, the plurality of protrusions 610 may be situated in a center portion of the spoked light transmission substrate 602. The plurality of protrusions 610 may be arranged in a circular pattern at the center portion of the spoked light transmission substrate 602. The plurality of protrusions 610 may extend inward from the circular contour surrounding the center portion of the spoked light transmission substrate 602. Each protrusion of the plurality of protrusions 610 may include an inner side extending away from the circular contour and towards the center. Each protrusion of the plurality of protrusions 610 may include an outer side coupled to an edge of the circular contour. The center portion of the spoked light transmission substrate 602 may be configured to align with the plurality of light emitters 104 and the light sensor at the back of the wearable biometric tracker 102.

The plurality of protrusions 610 may be configured to cover at least one of the plurality of light emitters 104, the light sensor, the electrical heart sensor, the blood oxygen sensor, and the optical heart sensor. The spoked light transmission substrate 602 may be configured to have different stack heights based on the amount of resin poured. The height of the transparent resin may be based on light transmission needs, light sensor size and shape, light emitter size and shape, the type of wearable biometric tracker 102, and the wearable biometric tracker enclosure 302. The area between the protrusions may prevent the collection of water, steam, and sweat. In some embodiments, the spoked light transmission substrate 602 may be a cured semi-translucent epoxy resin. The plurality of protrusions 610 may be referred to as spokes in which each spoke of the plurality of spokes is configured to cover at least one of the plurality of light emitters 104, the light sensor, the electrical heart sensor, the blood oxygen sensor, and the optical heart sensor. For example, the spoked light transmission substrate 602 may have eight spokes to cover each light emitter of the plurality of light emitters 104.

In some embodiments, the spoked light transmission substrate 602 may be a covering of the plurality of light emitters 104 and the light sensor configured to attach to the back of the wearable biometric tracker 102. The spoked light transmission substrate 602 may include a plurality of apertures in the negative space between the plurality of light emitters 104 and the light sensor to enhance the breathability of the device. Enhancing the breathability of the device avoids water, steam, and sweat entrapment. The plurality of apertures at the negative space between the plurality of light emitters 104 and the light sensor allows for light to pass through the spoked light transmission substrate 602. In another example, the spoked light transmission substrate 602 may be formed at the portions aligning with the light emitters and the light sensor situated at the wearable biometric tracker 102.

FIG. 6C shows an example of the spoked light transmission substrate 602 superimposed over the backside of the wearable biometric tracker 102. The center portion of the domed light transmission substrate 106 may be configured to align with the plurality of light emitters 104 and the light sensor at the back of the wearable biometric tracker 102. The plurality of protrusions 610 may be configured to cover at least one of the plurality of light emitters 104, the light sensor, the electrical heart sensor, the blood oxygen sensor, and the optical heart sensor. The plurality of protrusions 610 may be referred to as spokes in which each spoke of the plurality of spokes is configured to cover at least one of the plurality of light emitters 104, the light sensor, the electrical heart sensor, the blood oxygen sensor, and the optical heart sensor. For example, the spoked light transmission substrate 602 may have eight spokes to cover each light emitter of the plurality of light emitters 104.

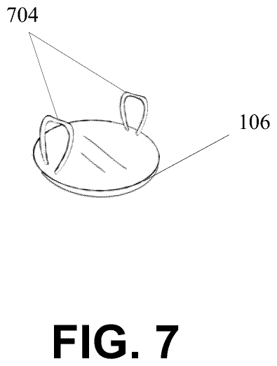

FIG. 7 depicts an example of a detachable spoked light transmission substrate 602 configured to cover the light sensor and the plurality of light emitters 104 using a pair of loops. The detachable spoked light transmission substrate 602 may include a pair of loops 704 coupled to the edges of the detachable spoked light transmission substrate 602. The pair of loops 704 can be selectively attached to the straps or wristbands coupled to the wearable biometric tracker 102. This design allows for a greater assortment of wearable biometric trackers 102 to benefit from the light transmissive properties of the detachable spoked light transmission substrate 602. In some embodiments, the pair of loops 704 can be easily disengaged from the wristbands of the wearable biometric tracker 102 when charging or recharging the wearable biometric tracker 102. In some embodiments, the spoked light transmission substrate 602 may be thicker at the portions covering the light sensor and the light emitters 104 relative to other portions of the spoked light transmission substrate 602 covering other portions of the back portion 100.

In some embodiments, the detachable spoked light transmission substrate 602 may be convex or have a dome shape at the portion covering the light sensor and the plurality of light emitters 104. In some embodiments, the spoked light transmission substrate 602 may be made of two materials in which a first material covers the light sensor and a second material covers the plurality of light emitters 104.

The detachable spoked light transmission substrate 602 may be translucent and formed of one or more of silicone, polycarbonate, polymethyl methacrylate (PMMA), epoxy resin, glass, and other comparable substances. The detachable spoked light transmission substrate 602 may have a circular or spherical shape and may include a convex or concave geometry. In some aspects, the detachable spoked light transmission substrate 602 may be formed in a number of other shapes, (e.g., rectangle, square), including a shape that conforms to the back portion 100 of a particular wearable biometric tracker 102, and so forth. Further, the spoked light transmission substrate 602 can have a viscosity value in a range of 500 to 10,000 centipoise (cps).

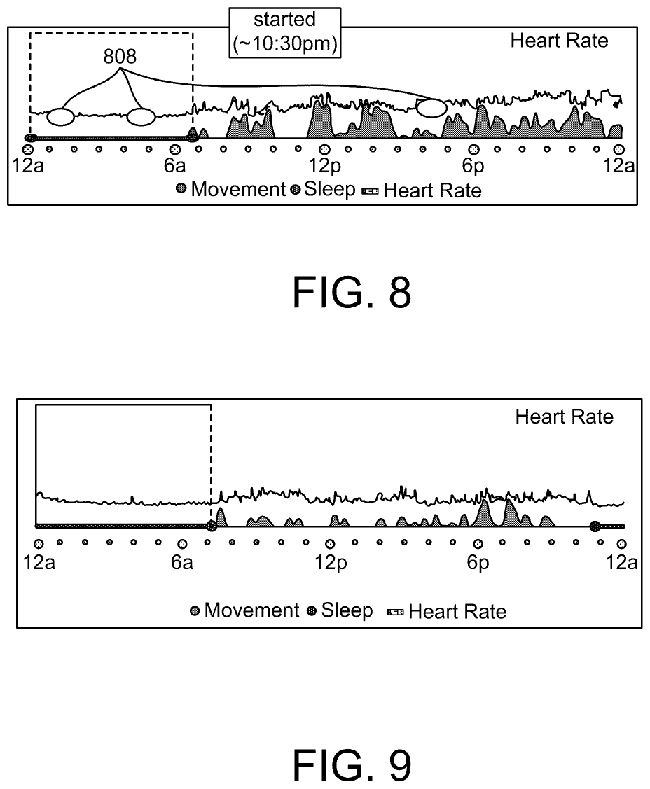

FIG. 8 depicts a user interface that includes data obtained by various sensors of a fitness tracker without applying the light transmission substrate. FIG. 8 depicts data capture gaps 808 due to the failed transmission of light at the skin interface between the user skin and the wearable biometric tracker 102. The failed transmission of light at the skin interface may be due to tattoos, dark skin obstructions, excessive body hair or because the user is an obese individual. The failed transmission of light may be due to other interference with one or more PPG signals emitted by the light source of the fitness tracker (e.g., Garmin smartwatch). The data capture gaps 808 in the heart rate are indicated by circles included at the user interface.

FIG. 9 depicts another user interface depicting data obtained by various sensors of a fitness tracker with the application of the light transmission substrate. As shown in FIG. 9, the use of the light transmission substrate enables obtaining continuous readings of heart rate data over a prolonged period of time such that there are few or negligible data acquisition gaps.

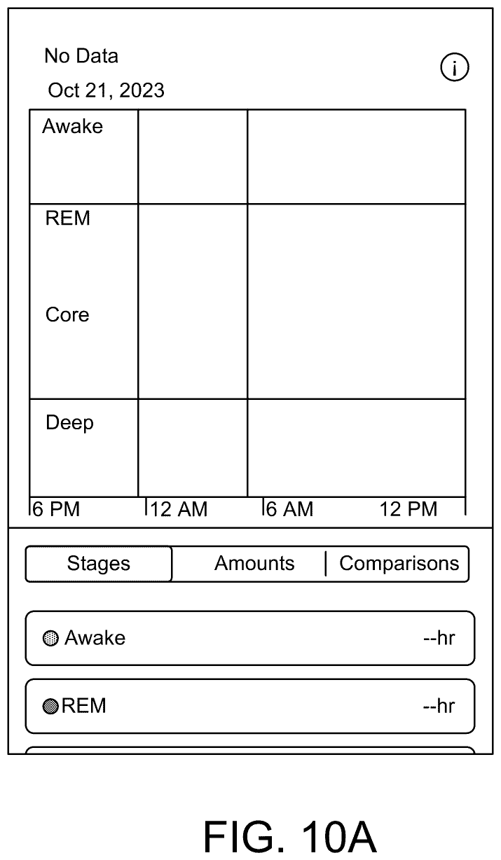

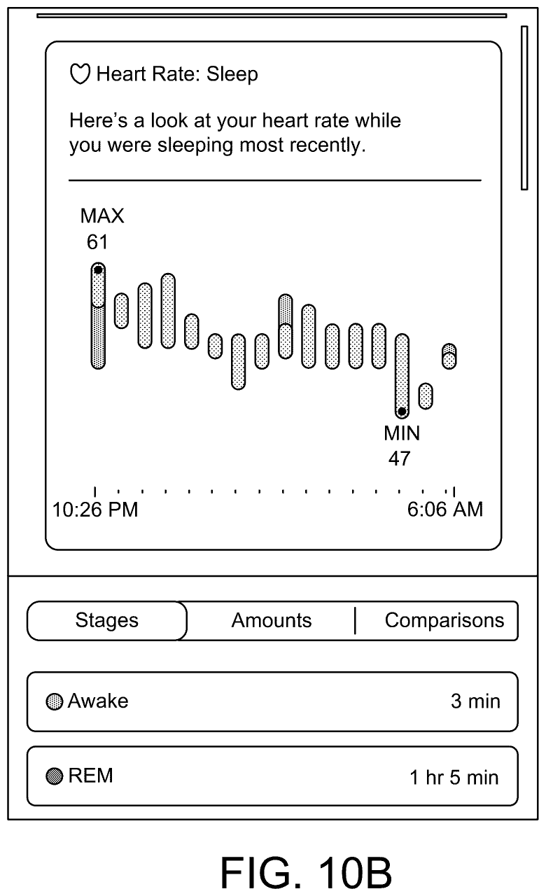

FIGS. 10A and 10B depict user interfaces that includes a comparison of the operation of a fitness tracker without the light transmission substrate to the operation of the fitness tracker with the light transmission substrate. As shown in FIG. 10A, the fitness tracker without the application of light transmission substrate resulted in a data acquisition gap of approximately 4 hours. Specifically, the fitness tracker sporadically captured heart rate data at various time periods without the light transmission substrate.

FIG. 10B depicts a user interface that shows the manner in which the fitness tracker operates when applying the light transmission substrate and/or the wearable biometric tracker enclosure 302. In contrast to the fitness tracker without the domed light transmission substrate 106, the fitness tracker obtained continuous readings with only minor gaps in data acquisition with the application of the wearable biometric tracker enclosure 302. As shown in FIG. 10B, the fitness tracker used with the light transmission substrate resulted in a nearly continuous acquisition of data for approximately 8 hours from 10:26 PM to 6:06 AM.

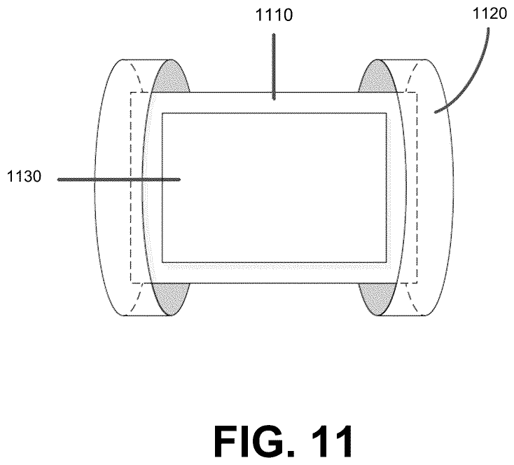

FIG. 11 depicts a top view of an optical lens casing 1110 that partially encloses a layered light transmission substrate 1130. The optical lens casing 1110 may be configured to house the layered light transmission substrate 1130. The optical lens casing 1110 may be configured to suspend the layered light transmission substrate 1130 over the light sensor at the fitness tracker and the plurality of light emitters 104 using a pair of elastic loops 1120. The optical lens casing 1110 may be coupled to a pair of elastic loops 1120 at the ends of the optical lens casing 1110. The pair of elastic loops 1120 may be coupled at opposing ends of the optical lens casing 1110. The pair of elastic loops 1120 may be parallel to each other. The pair of elastic loops 1120 may be parallel to opposing ends of the optical lens casing 1110. The pair of elastic loops 1120 may be parallel to the same opposing ends of the optical lens casing 1110 to which the pair of elastic loops 1120 are attached. The pair of elastic loops 1120 may connect at a top surface, a bottom surface, or a side surface of the optical lens casing 1110. The optical lens casing 1110 can be selectively attached to the straps or wristbands coupled to the wearable biometric tracker 102. This design allows for a greater assortment of wearable biometric trackers 102 to benefit from the light transmissive properties of the layered light transmission substrate 1130. In some embodiments, the pair of elastic loops 1120 can be easily disengaged from the wristbands of the wearable biometric tracker 102 when charging or recharging the wearable biometric tracker 102.

The optical lens casing 1110 may partially enclose the light transmission substrate 106. The optical lens casing 1110 may cover the outer edges of the layered light transmission substrate 1130 while leaving an exposed center portion of the layered light transmission substrate 1130. The layered light transmission substrate 1130 may be sandwiched between two layers of the optical lens casing 1110 at the edges of the layered light transmission substrate 1130. In some embodiments, the layered light transmission substrate 1130 may be thicker at its exposed center portion in comparison to the edges that are sandwiched between two layers of the optical lens casing 1110. The exposed center portion of the layered light transmission substrate 1130 may be configured to cover the light sensor and the light emitters 104 while the outer edges of the layered light transmission substrate 1130 are sandwiched between two layers of the optical lens casing 1110 that surrounds the light sensor and the light emitters 104.

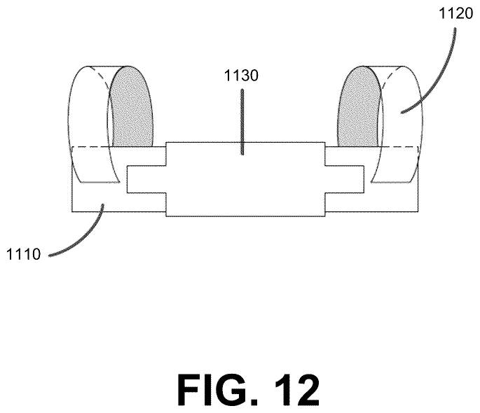

FIG. 12 depicts a side view of an optical lens casing 1110 having elastic loops 1120 that partially encloses a layered light transmission substrate 1130. The optical lens casing 1110 may enclose opposing ends of the layered light transmission substrate 1130. The optical lens casing 1110 may surround at least a portion of the layered light transmission substrate 1130. The optical lens casing 1110 may be thicker than opposing ends of the layered light transmission substrate 1130. The optical lens casing 1110 may cover an upper surface and a lower surface of the opposing ends of the layered light transmission substrate 1130. The optical lens casing 1110 may operate to position the layered light transmission substrate 1130 (using the loops) over the biometric sensor and the plurality of lights for consistent and steady placement.

In some embodiments, the layered light transmission substrate 1130 may be thicker than the optical lens casing 1110 at the unexposed center portion of the layered light transmission substrate 1130. The unexposed center portion of the layered light transmission substrate 1130 may “poke out” of the optical lens casing 1110 to allow for closer contact between the smartwatch and the skin. The unexposed center portion of the layered light transmission substrate 1130 may be thicker than the opposing ends of the layered light transmission substrate 1130 that are sandwiched by the optical lens casing 1110. The elastic loops 1120 may be coupled to the side edges of the optical lens casing 1110. Additionally, and/or alternatively, the pair of elastic loops 1120 may connect at a top surface or a bottom surface of the optical lens casing 1110. The elastic loops may be made out of polyester rubber, string, fabric, strap, tie, clip, magnet, plastic, metal clasp, velcro, and/or the like. The optical lens casing 1110 may be made out of waterproof woven nylon (coated with polyurethane), neoprene, bamboo, canvas, vinyl, polyester, cotton, PVC, acrylic, polypropylene, and/or the like.

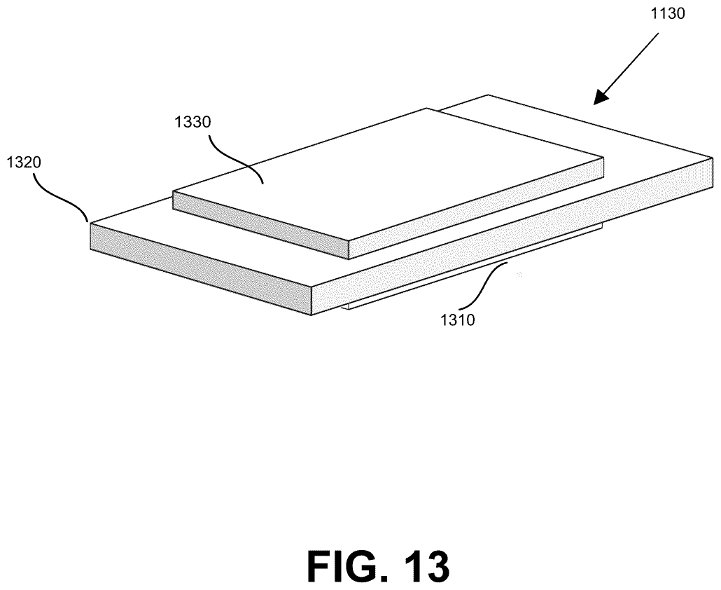

FIG. 13 depicts the layered light transmission substrate 1130 configured to be coupled to the partially enclosed by the optical lens casing 1110 having elastic loops 1120. The layered light transmission substrate 1130 may include three layers: a bottom substrate layer 1310, a center substrate layer 1320, and a top substrate layer 1330. The center substrate layer 1320 may be situated between the bottom substrate layer 1310 and the top substrate layer 1330. The center substrate layer 1320 may have a larger footprint than the top substrate layer 1330 and the bottom substrate layer 1310. In some embodiments, the top substrate layer 1330 and the bottom substrate layer 1310 may have the same-sized footprint. In some embodiments, the center substrate layer 1320 may be thicker than the top substrate layer 1330 and the bottom substrate layer 1310.

The bottom substrate layer 1310, the center substrate layer 1320, and the top substrate layer 1330 may create a domed effect that amplifies the range of the transmitted light signal from the plurality of emitters. The layered light transmission substrate 1130 may have a circular or spherical shape and may include a convex or concave geometry. In some aspects, the layered light transmission substrate 1130 may be formed in a number of other shapes, (e.g., rectangular, square, hexagon, trapezoid, rhombus, with or without spokes), including a shape whose height is sloped, flat, or hard ridged. The shape whose height is sloped, flat, or hard ridged may support the protruding shape of the layered light transmission substrate 1130 from the optical lens casing 1110. The corners may be rounded or straight.

The layered light transmission substrate 1130 may be a clear PVC resin base and clear epoxy. The layered light transmission substrate 1130 may be UV resistant to reduce yellowing and degradation. The layered light transmission substrate 1130 may be translucent and formed of one or more of silicone, polycarbonate, polymethyl methacrylate (PMMA), epoxy resin, glass, and other comparable substances. Further, the layered light transmission substrate 1130 may have a viscosity value in a range of 500 to 10,000 centipoise (cps).

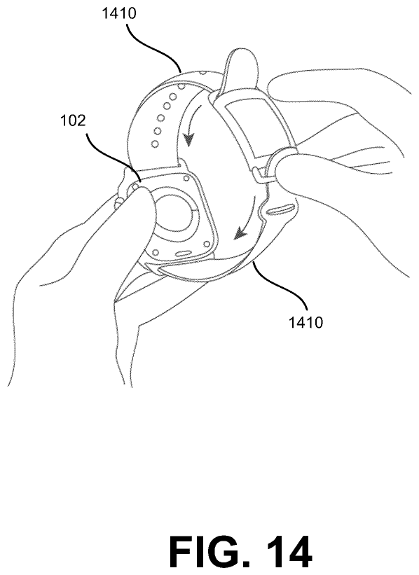

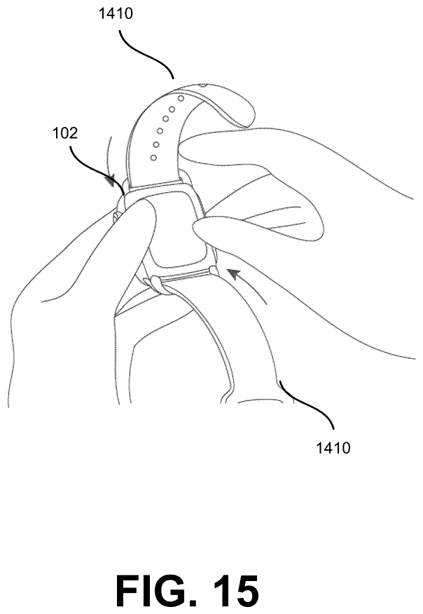

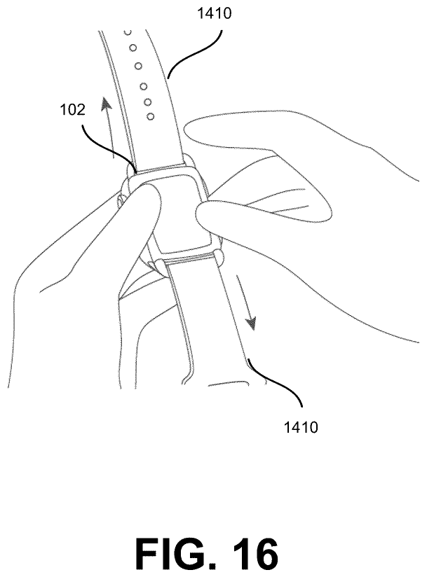

FIGS. 14-16 depict a method of applying the optical lens casing 1110 to a wearable biometric tracker 102 having straps 1410. The straps 1410 may be coupled to opposing ends of the wearable biometric tracker 102. Each of the straps 1410 may have a closed side and an open side. The closed side may be configured to couple with the wearable biometric tracker 102. The open side may be opposite of the closed side of the strap 1410 and may selectively attach to the other strap.

The open side of each strap 1410 of the wearable biometric tracker 102 may be inserted into each loop of the pair of elastic loops 1120. If necessary, the opening formed by each loop of the pair of elastic loops 1120 may be enlarged to allow insertion of the open side of the strap. Each strap 1410 of the wearable biometric tracker 102 may be pulled through each loop of the pair of elastic loops 1120 until each loop reaches the closed side of each strap 1410 connecting to the wearable biometric tracker 102. At the closed side of each strap 1410, the optical lens casing 1110 may be situated such that the exposed portion of the layered light transmission substrate 1130 covers the light sensor and the plurality of emitters 104 at the wearable biometric tracker 102. In some embodiments, the straps 1410 may be pulled away from each other in opposite directions to increase the likelihood that the optical lens casing 1110 and the pair of loops 1120 are properly situated over the light sensor and the plurality of emitters 104.

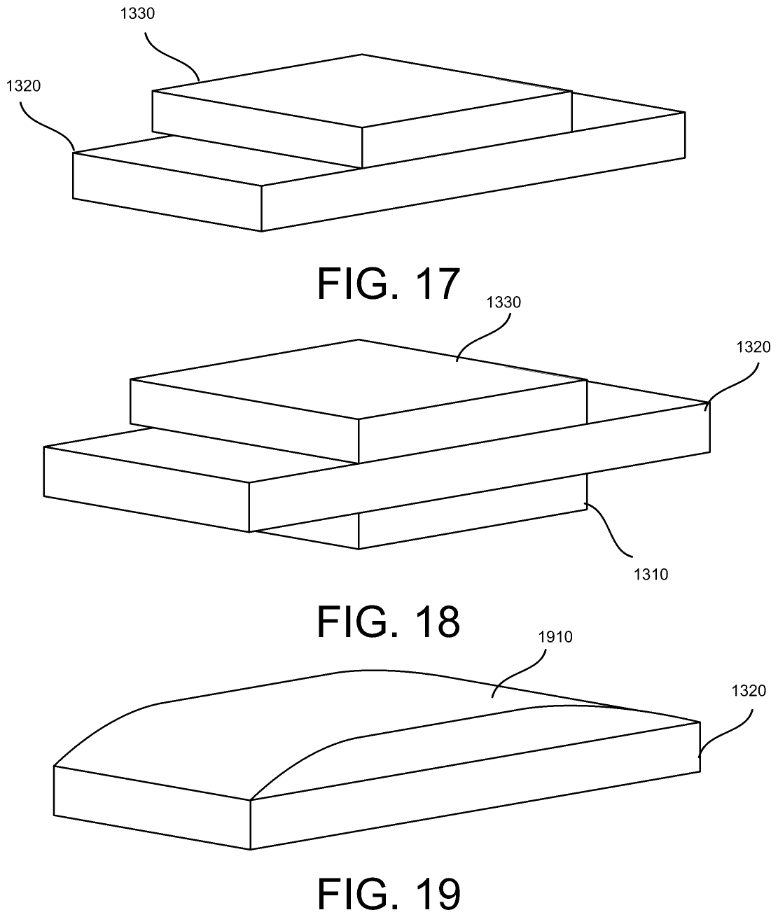

FIG. 17 depicts another light transmission substrate configured to be coupled to the partially enclosed by the optical lens casing 1110 having elastic loops 1120. The layered light transmission substrate 1130 may include two layers: a center substrate layer 1320 and a top substrate layer 1330. The center substrate layer 1320 may be situated above the top substrate layer 1330. The center substrate layer 1320 may have a larger footprint than the top substrate layer 1330. In some embodiments, the center substrate layer 1320 may be the same thickness as the top substrate layer 1330. In some embodiments, the top substrate layer 1330 may be 16 mm by 24 mm by 1.5 mm. In some embodiments, the center substrate layer 1320 may be 20 mm by 30 mm by 1.5 mm. The total thickness of the two layers may be 3.0 mm.

FIG. 18 depicts another light transmission substrate configured to be coupled to the partially enclosed by the optical lens casing 1110 having elastic loops 1120. The layered light transmission substrate 1130 may include three layers: a bottom substrate layer 1310, a center substrate layer 1320, and a top substrate layer 1330. The center substrate layer 1320 may be situated between the bottom substrate layer 1310 and the top substrate layer 1330. The center substrate layer 1320 may have a larger footprint than the top substrate layer 1330 and the bottom substrate layer 1310. In some embodiments, the top substrate layer 1330 and the bottom substrate layer 1310 may have the same sized footprint. In some embodiments, the center substrate layer 1320 may be thicker than the top substrate layer 1330 and the bottom substrate layer 1310. In some embodiments, the top substrate layer 1330 may be 16 mm by 24 mm by 1.0 mm. In some embodiments, the center substrate layer 1320 may be 20 mm by 30 mm by 1.0 mm. In some embodiments, the bottom substrate layer 1310 may be 16 mm by 24 mm by 1.0 mm. The total thickness of the three layers may be 3.0 mm.

FIG. 19 depicts another light transmission substrate configured to be coupled to the partially enclosed by the optical lens casing 1110 having elastic loops 1120. The layered light transmission substrate 1130 may include two layers: a center substrate layer 1320 and a sloped top substrate layer 1910. The center substrate layer 1320 may be situated above the sloped top substrate layer 1910. The center substrate layer 1320 may have the same footprint as the bottom surface of the sloped top substrate layer 1910. The sloped top substrate layer 1910 may be 1.5 mm. In some embodiments, the center substrate layer 1320 may be thicker than the top substrate layer 1330. In some embodiments, a top surface of the top substrate layer 1330 may be 16 mm by 24 mm. The thickness of the top substrate layer 1330 may be 1.5 mm. In some embodiments, the center substrate layer 1320 may be 20 mm by 30 mm by 1.5 mm. The total thickness of the two layers may be 3.0 mm.

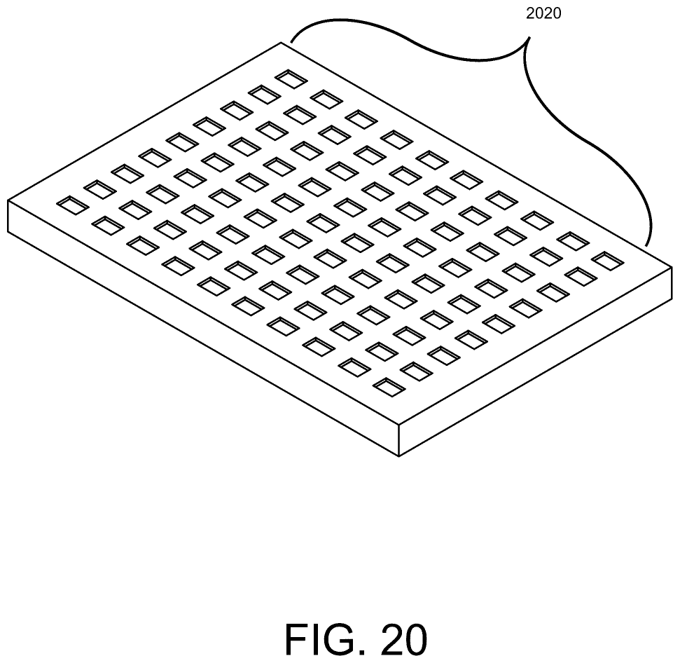

FIG. 20 depicts a perspective view of a tray having a plurality of mold wells 2020 configured to form a plurality of light transmission substrates. The tray may be rectangular and have the plurality of mold wells 2020 arranged in a grid-like manner across a top surface of the tray. The tray may have a width and a length. The mold wells 2020 of the plurality of mold wells 2020 may be arranged into rows extending the length of the tray and columns extending the width of the tray.

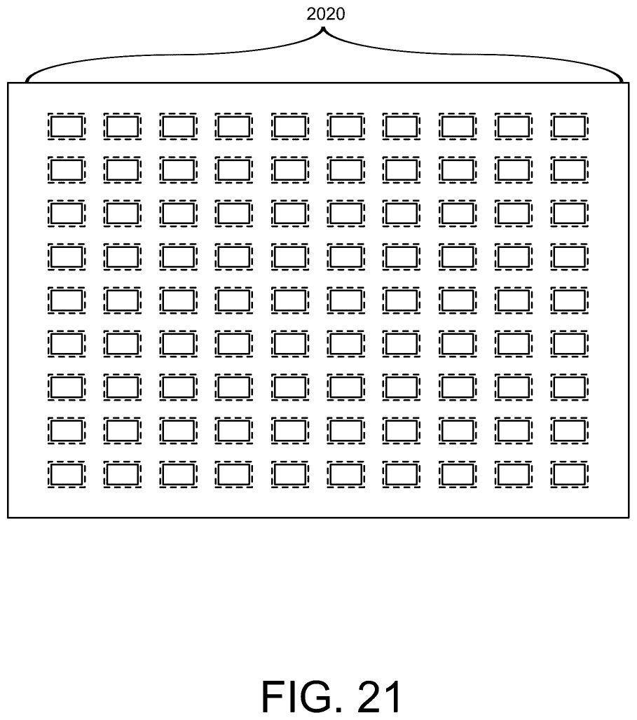

FIG. 21 depicts a perspective view of a tray having a plurality of mold wells 2020 configured to form a plurality of light transmission substrates. The tray may be rectangular and have the plurality of mold wells 2020 arranged in a grid-like manner across a top surface of the tray. The tray may have a width and a length. The mold wells 2020 of the plurality of mold wells 2020 may be arranged into rows extending the length of the tray and columns extending the width of the tray. The length of the tray may be 500 mm and the width of the tray of 350 mm. The distance between long edge of the tray and the closest row of molds wells may be 25 mm. The distance between the short edge of the tray and the closest column of mold wells is 32.5 mm. Each of the mold wells 2020 may be spaced 15 mm away from the nearest mold well.

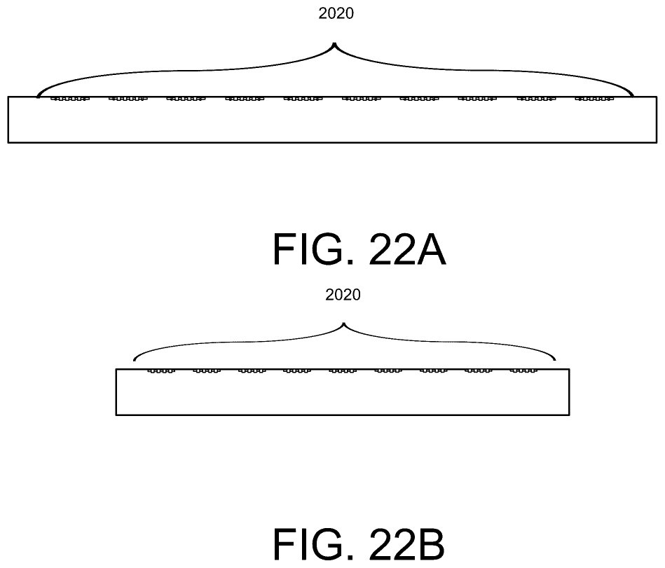

FIG. 22A depicts a cross-sectional view of the tray in which the cross-section is made along the length of the tray. Each mold well of the plurality of mold wells 2020 has a depth that partially extends into the tray.

FIG. 22B depicts a cross-sectional view of the tray in which the cross-section is made along the width of the tray. Each mold well of the plurality of mold wells 2020 has a depth that partially extends into the tray.

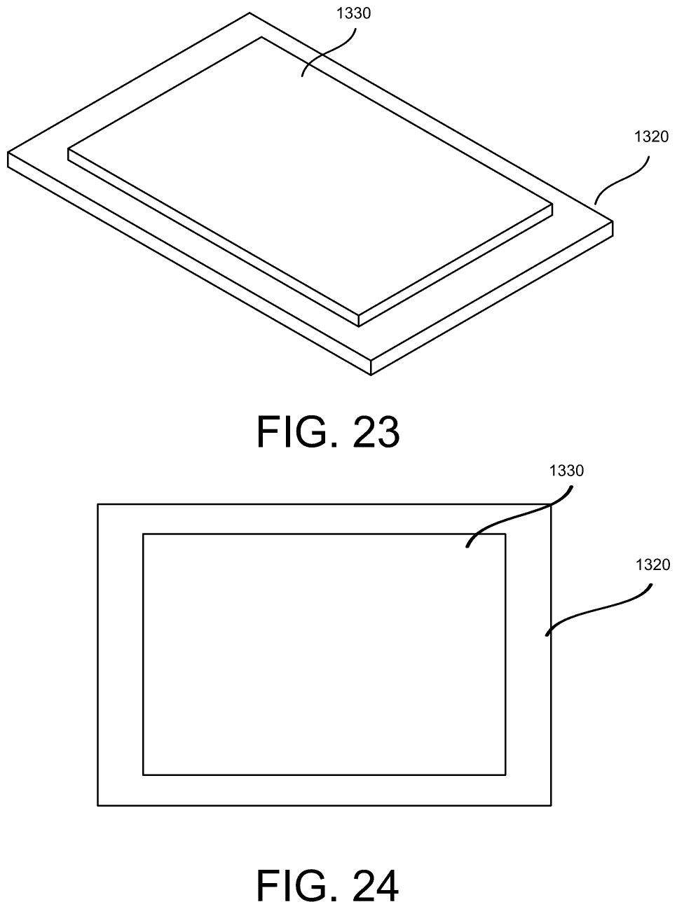

FIG. 23 depicts a perspective view of a three-layered light transmission substrate. The three-layered layered light transmission substrate 1130 may include three layers: a bottom substrate layer 1310, a center substrate layer 1320, and a top substrate layer 1330. The center substrate layer 1320 may be situated between the bottom substrate layer 1310 and the top substrate layer 1330. The center substrate layer 1320 may have a larger footprint than the top substrate layer 1330 and the bottom substrate layer 1310. In some embodiments, the top substrate layer 1330 and the bottom substrate layer 1310 may have the same-sized footprint. In some embodiments, the center substrate layer 1320 may be thicker than the top substrate layer 1330 and the bottom substrate layer 1310.

FIG. 24 depicts a top view of a three-layered light transmission substrate. The center substrate layer 1320 may have the dimensions of 20 mm by 30 mm, the top substrate layer 1330 may have the dimensions of 16 mm by 24 mm, and the bottom substrate layer 1310 may have the dimensions of 16 mm by 24 mm.

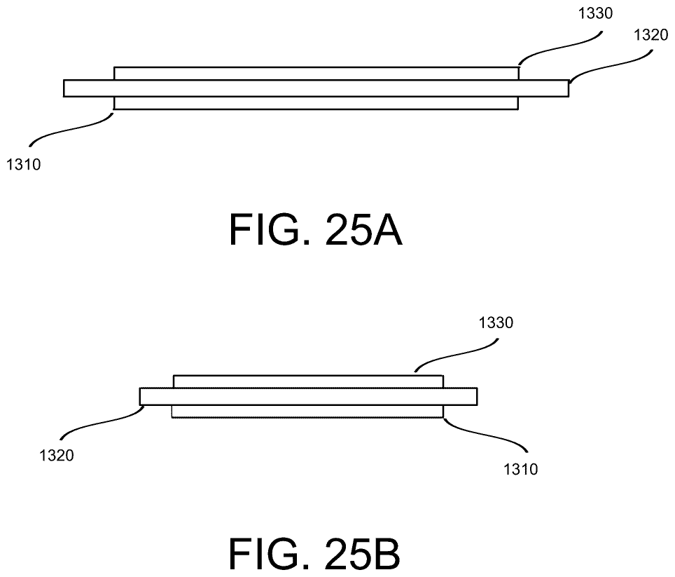

FIG. 25A depicts a side view of a three-layered light transmission substrate along a length of the light transmission substrate. The bottom substrate layer 1310 and the top substrate layer 1330 may have a thickness of 0.75 mm.

FIG. 25B depicts a side view of the three-layered light transmission substrate along a length of the light transmission substrate. The center substrate layer 1320 may have a thickness of 1.0 mm. The total thickness of the three-layered light transmission substrate may be 2.5 mm.

The terminology used herein is for the purpose of describing particular embodiments only and is not intended to be limiting of the embodiments. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

1. A transparent resin comprising:
a first side and a second side opposite the first side, the first side configured to cover a plurality of light emitters and a light sensor at a wearable biometric tracker, the first side configured to receive light from the plurality of light emitters at the wearable biometric tracker, the second side configured to interface with a user skin tissue, the second side configured to pass light received by the transparent resin to the user skin tissue,
wherein the transparent resin is configured to pass light from the plurality of light emitters to the user skin tissue with the transparent resin positioned between the wearable biometric tracker and the user skin tissue.

2. The transparent resin of claim 1, wherein the transparent resin has a viscosity value in a range of 500 to 10,000 centipoise (cps).

3. The transparent resin of claim 1, wherein the transparent resin has a greater thickness at an inner portion covering the plurality of light emitters and the light sensor than an outer portion of the transparent resin.

4. The transparent resin of claim 3, wherein the inner portion is made of a first material and wherein the outer portion are made of a second material.

5. The transparent resin of claim 1, wherein the second side is configured to receive at least one of reflected light or transmitted light from the user skin tissue and the first side is configured to pass reflected light received by the user skin tissue to the wearable biometric tracker,
wherein the transparent resin is configured to pass the reflected light from the user skin tissue to the light sensor of the wearable biometric tracker with the transparent resin situated between the wearable biometric tracker and the user skin tissue.

6. The transparent resin of claim 1, wherein the second side of the transparent resin has at least one of a convex geometry or a dome-shaped protrusion.

7. The transparent resin of claim 1, wherein the transparent resin has an index of refraction in a range of approximately 1.4 to 1.7.

8. The transparent resin of claim 1, wherein the transparent resin has an index of refraction in a range of approximately 1.5 to 1.56.

9. The transparent resin of claim 1, wherein the light sensor of the wearable biometric tracker is a photoplethysmography (PPG) sensor and an LED is at least one light emitter of the plurality of light emitters.

10. The transparent resin of claim 1, wherein the transparent resin is configured to perform at least one of refract or redirect light passing through the transparent resin.

11. A device comprising:
a light transmission substrate including two or more transparent layers with a first transparent layer disposed over a second transparent layer, the first transparent layer configured to cover a plurality of light emitters and a light sensor at a wearable biometric tracker, the first transparent layer configured to receive light from the plurality of light emitters at the wearable biometric tracker, the second transparent layer configured to interface with a user skin tissue, the second transparent layer configured to pass light received by the light transmission substrate to the user skin tissue; and
a casing configured to suspend the two or more transparent layers between the wearable biometric tracker and the user skin tissue,
wherein the light transmission substrate is configured to pass light from the plurality of light emitters to the user skin tissue with the light transmission substrate positioned between the wearable biometric tracker and the user skin tissue.

12. The device of claim 11, wherein the casing further comprises two or more elastic loops configured to suspend the two or more transparent layers between the wearable biometric tracker and the user skin tissue.

13. The device of claim 12, wherein the two or more elastic loops are situated at opposing ends of the casing.

14. The device of claim 12, wherein the two or more elastic loops are parallel to each other.

15. The device of claim 13, wherein the two or more elastic loops are parallel to each other and to the opposing ends of the casing.

16. The device of claim 11, wherein the casing covers an outer edge of the light transmission substrate while leaving an exposed center portion of the light transmission substrate.

17. The device of claim 16, wherein one transparent layer of the two or more transparent layers has a height dimension that extends beyond the casing.

18. The device of claim 17, wherein the other transparent layer of the two or more transparent layers has the outer edge covered by the casing.

19. The device of claim 11, wherein the light transmission substrate includes three or more transparent layers.

20. The device of claim 11, wherein the second transparent layer is configured to receive at least one of reflected light or transmitted light from the user skin tissue and the first transparent layer is configured to pass reflected light received by the user skin tissue to the wearable biometric tracker,
wherein the light transmission substrate is configured to pass the at least one of the reflected light or the transmitted light from the user skin tissue to the light sensor of the wearable biometric tracker with the light transmission substrate situated between the wearable biometric tracker and the user skin tissue.
